SEQUENCE LISTING (18 sequences)

SEQ ID NO: 1 - protein, 887 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKT

SEQ ID NO: 2 - protein, 965 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGAL

SEQ ID NO: 3 - protein, 1038 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQ

SEQ ID NO: 4 - protein, 1142 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQI

SEQ ID NO: 5 - protein, 1211 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTAT

SEQ ID NO: 6 - protein, 1296 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEF

SEQ ID NO: 7 - protein, 1304 aa, Artificial sequence
  Synthetic construct
  Synthetic construct
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTKAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGADKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVDWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSERVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERD

SEQ ID NO: 8 - protein, 1931 aa, Artificial sequence
  Synthetic construct
  Synthetic construct
MKKPLGKIVASTALLISVAFSSSIASAAVEDATRSDSTTQMSSTPEVAYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQLNYDDDSTGTVTWNTQTAGDFTFKPALDAKLKVTDKTVTVTYQGKSAVIDITVSQPAPTVSKTDLDKAIKAIEAKNPDSSKYTADSWKTFADAMAHAKAVIADDSATQQDVDNALKALTDAYAGLTEKTPEPAPVSKSELDKKIKAIEAEKLDGSKYTAESWKAFETALAHAKAVIASDSATQQNVDAALGALTSARDGLTEKGEVKPDPKPEPGTVDKAALDKAVKKVEAEKLDGSKYTADSWKAFETALAHAKAVIGNANSTQFDIDNALSMLNDARAALKEKPGRIIAIIDGSALSKTGASVAIIASVAAAMLAVGAGVMALRRKRS

SEQ ID NO: 9 - protein, 1341 aa, Artificial sequence
  Synthetic construct
  Synthetic construct
MKKPLGKIVASTALLISVAFSSSIASAIEDATRSDSTTQMSSTPEVAYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFSATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEQRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVRVTVTNADTTTPSGVVCAGLTEIELKTATSKFVANTSAALSSLTVNGTKVSDSVLAAGSYNTPAIIADVKAEGEGNASVTVLPAHDNVIRVITESEDHVTRKTFTINLGTEQEFPADSDERDQHQHQHQHQQ

SEQ ID NO: 10 - protein, 1752 aa, Artificial sequence
  Synthetic construct
  Synthetic construct
MAVRRLGGRIVAFAATVALSIPLGLLTNSAWAVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQHDRQADRPPQPLVGEQADERGLTCGTRCDRVEQLRKHENREAHRTGLDHLEFVGAADGAVGEQATFKVHVHADQGDGRHDDADERDIDPHVPVDHAVGELARAACHHVIGLRVDTHRLKASGFQIPADDMAEIDRITGFHRFERHVG

SEQ ID NO: 11 - protein, 1935 aa, Artificial sequence
  synthetic construct
  synthetic construct
MAVRRLGGRIVAFAATVALSIPLGLLTNSAWAVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTKAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGADKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVDWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTAGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQLNYDDDSTGTVTWNTQTAGDFTFKPALDAKLKVTDKTVTVTYQGKSAVIDITVSQPAPTVSKTDLDKAIKAIEAKNPDSSKYTADSWKTFADAMAHAKAVIADDSATQQDVDKALKALTDAYAGLTEKTPEPAPVSKSELDKKIKAIEAEKLDGSKYTAESWKAFETALAHAKAVIASDSATQQDVDAALGALTSARDGLTEKGEVKPDPKPEPGTVDKAALDKAVKKVEAEKLDGSKYTADSWKAFETALAHAKAVIGNANSTQFDIDNALSMLNDARAALKEKPGRIIAIIDGGALSKTGASVAIIASVAAAMKAVGAGVMALRPPKW

SEQ ID NO: 12 - protein, 887 aa, Bifidobacterium bifidum; identical to SEQ ID NO: 1

SEQ ID NO: 13 - protein, 965 aa, Bifidobacterium bifidum; identical to SEQ ID NO: 2

SEQ ID NO: 14 - protein, 1038 aa, Bifidobacterium bifidum; identical to SEQ ID NO: 3

SEQ ID NO: 15 - protein, 1142 aa, Bifidobacterium bifidum; identical to SEQ ID NO: 4

SEQ ID NO: 16 - protein, 1211 aa, Bifidobacterium bifidum; identical to SEQ ID NO: 5

SEQ ID NO: 17 - protein, 1296 aa, Bifidobacterium bifidum; identical to SEQ ID NO: 6

SEQ ID NO: 18 - protein, 1720 aa, Bifidobacterium bifidum
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQHDRQADRPPQPLVGEQADERGLTCGTRCDRVEQLRKHENREAHRTGLDHLEFVGAADGAVGEQATFKVHVHADQGDGRHDDADERDIDPHVPVDHAVGELARAACHHVIGLRVDTHRLKASGFQIPADDMAEIDRITGFHRFERHVG

(19) United States
(12) Patent Application Publication
(10) Pub. No.: US 2024/0417765 A1
(43) Pub. Date: Dec. 19, 2024

(54) FERMENTED MILK-BASED PRODUCT COMPRISING GALACTO-OLIGOSACCHARIDES AND METHODS THEREOF

(71) Applicant: Chr. Hansen A/S, Hoersholm (DK)

(72) Inventors: Maria Elina Sundberg, Hoersholm (DK); Vojislav Vojinovic, Hoersholm (DK); Hans Raj, Hoersholm (DK)

(73) Assignee: Kerry Group Services International Ltd, Tralee (IE)

(21) Appl. No.: 18/549,668

(22) PCT Filed: Mar. 10, 2022
(86) PCT No.: PCT/EP2022/056189

§ 371 Date: Sep. 8, 2023

(30) Foreign Application Priority Data
Mar. 11, 2021 (EP) 21162010.9

(51) Int. Cl. C12P 19/04; A23C 9/12

(52) U.S. Cl. CPC C12P 19/04; A23C 9/1206; C12Y 302/01023

(57) ABSTRACT

The present invention relates to a fermented milk-based product comprising a combination of low levels of lactose and stable levels of galacto-oligosaccharides (GOS) produced in-situ and methods thereof.

### CROSS-REFERENCE TO RELATED APPLICATIONS

The present application is the U.S. National Stage of International Application No. PCT/EP2022/056189, filed Mar. 10, 2022, and claims priority to European Patent Application No. 21162010.9, filed Mar. 11, 2021.

### SEQUENCE LISTING

The instant application contains a Sequence Listing which has been submitted electronically in ASCII format and is hereby incorporated by reference in its entirety. Said ASCII copy, created on Apr. 18, 2024, is named “030427-0403_SL.txt” and is 192,408 bytes in size.

### FIELD OF THE INVENTION

The present invention relates to a fermented milk-based product, preferably a post-pasteurized fermented milk product, comprising a combination of low levels of lactose and stable levels of galacto-oligosaccharides (GOS) produced in situ by a beta-galactosidase and wherein GOS are stable over time and at room temperature. This fermented milk-based product can be consumed by an individual with reduced tolerance to food lactose while simultaneously stimulating the proliferation of beneficial colonic microorganisms, such as bacteria, to give physiological benefits to the individual.

The fermented milk-based product comprising GOS is obtained by a method comprising a step of inactivating the beta-galactosidase enzyme by a combination of heat treatment at a low pH. The inactivation of the enzyme avoids the degradation of the GOS previously formed. Thus, this invention also relates to the method for producing the fermented milk-based product herein disclosed.

### BACKGROUND OF THE INVENTION

### Galacto-Oligosaccharides (GOS)

GOS are non-digestable carbohydrates produced by the enzymatic treatment of lactose, by a beta-galactosidase. This enzymatic reaction produces a mixture of oligosaccharides of varying lengths linked by glycosidic bonds, unreacted lactose, and monomeric sugars (D-glucose and D-galactose).

GOS has the beneficial effect of acting as prebiotics by enhancing the growth and selectively stimulating the proliferation and activity of beneficial colonic microorganisms, such as bacteria from the genus Bifidobacterium, to give physiological benefits to the consumer, either at a short-, medium- or long-term. The genus Bifidobacterium is also associated to health-promoting effects, for example, ability to repopulate the intestinal flora in individuals who have had their intestinal flora disturbed by for example intake of antibiotics and/or ability to outcompete potential harmful intestinal micro-organisms.

The established health effects have contributed to the growing and continued interest in GOS as food ingredients for various types of food.

### Beta-Galactosidase Enzyme

The beta-galactosidase (EC 3.2.1.23) enzyme performs two different reactions, hydrolysis of lactose and transgalactosylation. In the first step, the glycosidic linkage between glucose and galactose in the lactose substrate is broken, and glucose is released and galactosyl enzyme intermediate is formed. In the second step, this new covalent bond in the galactosyl enzyme intermediate will be then attached by a nucleophile, either water or sugar. If the incoming nucleophile is water, then the resulting reaction is called hydrolysis and lead to the release of galactose. If the incoming nucleophile is another sugar, such as lactose, the resulting reaction is called galactosyl transfer reaction which leads to galacto-oligosaccharide formation (GOS formation). GOS formation by beta-galactosidase enzymes is favored, for example, by high concentrations of lactose or low water activity. Inactivation of beta-galactosidases in dairy applications is done by heat treatment or, alternatively, by applying low pH.

### Dairy Products

In recent years dairy products, for example fermented dairy products such as yogurts, which can be stored, transported, handled and consumed in non-refrigerated conditions, i.e. at ambient temperature, for several months have become widely used. Such products allow the consumer to carry the product with him/her for a period of time without the need for refrigeration, thereby providing a significant advantage for the consumer. In order to obtain such a long-term shelf life at ambient temperature, the fermented dairy product is heat-treated after completion of the fermentation process to at least inhibit further growth of the bulk of the lactic acid bacteria used in the fermentation process. The fermentation process captures all the benefits of bacterial cultures before the heat treatment process. The heat-treatment may e.g. be a pasteurization process or an Ultra High Temperature (UHT) process. Such products are sometimes referred to as post-pasteurized fermented milk products or ambient fermented milk products, in particular these products may be, for example, a Post-pasteurized Yogurt (PPY), also known as Ambient Yogurt.

Ambient yogurt, typically stirred or drinking yogurt, is therefore particularly relevant in regions where consumers are challenged by lack of cold chain conditions. Ambient yogurt reduces distribution costs and complexities compared to cold chain distribution, making it easier to export as the growing challenges on cold yogurt market are, all together, avoided.

The heat treatment process creates a product that can be stored at room temperature becoming a healthy and convenient alternative to cold yogurt, while still providing a good source of dairy protein, vitamins and minerals. However, ambient yogurt lack in situ generated GOS which are essential to give the above-mentioned physiological benefits to the consumer.

The health effects associated to GOS contributed to the its growing and continued interest as food ingredients for various types of food. However, it is of importance that the level or amount or concentration of GOS formed in or added to a product remains constant over time, in particular when stored at ambient temperature.

Several prior art documents disclose the in situ GOS production in milk and/or cold yogurt:

WO 2013/182686 suggests the synthesis of GOS in milk and cold yogurt. Example 4 of this patent document discloses the addition of a beta-galactosidase from Bifidobacterium bifidum to a milk-base simultaneous with addition of the specific yoghurt cultures, resulting in the trans-galactosylation reaction running together with the yoghurt fermentation process. The inactivation of the beta-galactosidase by a heat treatment of the milk and yogurt samples is carried out at 95° C. for 10 min. Fermented yogurt samples were stored at 4° C. to preserve the samples.

WO 2015/132402 relates to a method of producing non-fermented milk products containing GOS at low temperature, at most 10° C., using a beta-galactosidase enzyme from WO 2013/182686, and to a product containing GOS and monosaccharide sweeteners characterized in a weight ratio between glucose and galactose of at least 2:1. Further, the enzyme inactivation is carried out by a heat treatment of at least 140° C. for at least 0.1 second or alternatively at 95° C. for 10 min. As no fermentation step is carried out, the inactivation of the enzyme is made at a pH above 5.

WO 2020/117548 relates to a method for preparing a dairy product having a stable content of GOS fiber, and to a GOS fiber-enriched dairy product prepared by the method therein disclosed. In particular, WO 2020/117548 discloses a method for providing a low-lactose milk-based product having GOS fiber in which a milk substrate having lactose is treated with a beta-galactosidase enzyme derived from Bifidobacterium bifidum to provide GOS fiber and remaining lactose; deactivating the trans-galactosylating enzyme; contacting the milk-based substrate having GOS fiber with a lactase to degrade the remaining lactose to provide a low lactose milk-based product having GOS fiber and deactivating lactase. However, this patent document is silent about 1) using a single beta-galactosidase enzyme to both generate GOS and reduce lactose levels in fermented milk-based products, instead the method of WO 2020/117548 must use two different beta-galactosidase enzymes, a first to generate GOS and a second to degrade remaining lactose. Further, the enzyme generating GOS is inactivated (or deactivated) by heat treatment at 95° C. for 5 min prior to fermentation, and therefore at a pH above 5.

The prior art is, however, silent about methods for producing post-pasteurized fermented milk products or ambient fermented milk products enriched with stable levels of GOS produced in situ and with low levels of lactose, wherein the beta-galactosidase used for the GOS in situ produced is inactivated while simultaneously keeping the GOS produced in situ stable at room temperature over time in a fermented milk-based product that can be stored at room temperature.

In conclusion, there is a need for products having stable levels of in situ produced GOS and simultaneously low levels of lactose such that they can be consumed by a lactose intolerant individual, wherein said product is kept at ambient temperature without perish. There is also a need for a method for preparing said product.

### SUMMARY OF THE INVENTION

It is an object of the invention to provide a fermented milk-based product such a yogurt or yogurt product, comprising a combination of low levels of lactose and stable levels of galacto-oligosaccharides (GOS) produced in situ. This in situ GOS-comprising milk product or GOS-comprising milk-based product or in situ GOS-comprising yogurt or in situ GOS-comprising yogurt product can be stored at ambient temperature without perish and without compromising the stability of the GOS in the GOS-comprising milk product or GOS-comprising yogurt or GOS-comprising yogurt product, meaning that the in situ GOS, once produced, are stable over time.

It is further an object of the invention to provide a method for preparing or producing in situ GOS-comprising milk product or GOS-comprising milk-based product or in situ GOS-comprising yogurt or in situ GOS-comprising yogurt product herein disclosed.

The objects of the invention have been achieved when the inventors surprisingly found that it is possible to inactivate a beta-galactosidase enzyme, after the enzyme used lactose as a substrate to generate in situ GOS, if the enzyme is submitted to a heat treatment performed at a low pH, in particular wherein the heat treatment at low pH occurs at a temperature below 95° C., or below 90° C., in particular at a temperature from 70° C. to 85° C., and at a pH below 5, while simultaneously contributing to a reduction of energy needed to carry out the method. By inactivating the enzyme at a temperature below 95° C., or below 90° C., in particular at a temperature from 70° C. to 85° C., and at a pH below 5, it is possible to obtain or produce a low-lactose milk-based product, such as yogurt or yogurt product, enriched with in situ GOS, wherein GOS are stable over time as a result of the enzyme inactivation, and at ambient temperature. Finally, as lactose serves as the substrate for the beta-galactosidase enzyme, the final product has a reduced content of lactose, and as a result calorie reduction is enabled.

None of the cited prior art documents discloses a method for producing post-pasteurized fermented milk products or ambient fermented milk products enriched with stable levels of GOS produced in situ and with low levels of lactose, wherein the beta-galactosidase used for the GOS in situ produced is inactivated by a combination of a temperature below 95° C., in particular at a temperature from 70° C. to 85° C., and a pH below 5, while simultaneously keeping the GOS produced in situ stable at room temperature over time in a fermented milk-based product, such as a yogurt or a yogurt product, that can be stored at room temperature. In fact, the prior art is aligned in that the necessary conditions to inactivate a beta-galactosidase enzyme should be a temperature of at least 95° C., regardless of the pH.

### Method for Producing a Fermented Milk-Based Product, Preferably Yogurt or Yogurt Product, or More Preferably Ambient Yogurt, Comprising Galacto-Oligosaccharides

This invention relates to a method for producing a fermented milk-based product comprising galacto-oligosaccharides, wherein the method comprises the steps of:
(a) providing a milk-based substrate comprising lactose;
(b) treating the milk-based substrate with a beta-galactosidase enzyme to generate galacto-oligosaccharides followed by fermenting the milk-based substrate treated with the beta-galactosidase enzyme until a pH below 5 is reached, preferably at 43° C.; or alternatively fermenting the milk-based substrate until a pH below 5 is reached, preferably at 43° C., followed by treating the fermented milk-based substrate with a beta-galactosidase enzyme to generate galacto-oligosaccharides;
(c) inactivating the beta-galactosidase enzyme by submitting the fermented milk-based substrate comprising galacto-oligosaccharides having a pH below 5 to a temperature below 95° C., preferably from 70° C. to less than 95° C. or from 70° C. to 90° C., preferably from 70° C. to 85° C. or from 72° C. to 80° C., more preferably from 70° C. to 75° C., whereby producing the fermented milk-based product comprising galacto-oligosaccharides,
preferably wherein the galacto-oligosacharides comprised in the fermented milk-based product, such as yogurt or yogurt product, are stable for at least 5 days at a temperature of 15-37° C., preferably 15-30° C., more preferably 18-24° C., and at a pressure of 1 atm, or
preferably wherein the galacto-oligosacharides comprised in the fermented milk-based product, such as yogurt or yogurt product, are stable for at least 10 days at a temperature of 15-37° C., preferably 15-30° C., more preferably 18-24° C., and at a pressure of 1 atm, or
preferably wherein the galacto-oligosacharides comprised in the fermented milk-based product, such as yogurt or yogurt product, are stable for at least 15 days at a temperature of 15-37° C., preferably 15-30° C., more preferably 18-24° C., and at a pressure of 1 atm, or
preferably wherein the galacto-oligosacharides comprised in the fermented milk-based product, such as yogurt or yogurt product, are stable for at least 21 days at a temperature of 15-37° C., preferably 15-30° C., more preferably 18-24° C., and at a pressure of 1 atm, or
preferably wherein the galacto-oligosacharides comprised in the fermented milk-based product, such as yogurt or yogurt product, are stable for at least 90 days at a temperature of 15-37° C., preferably 15-30° C., more preferably 18-24° C., and at a pressure of 1 atm, or
preferably wherein the galacto-oligosacharides comprised in the fermented milk-based product, such as yogurt or yogurt product, are stable for at least 180 days at a temperature of 15-37° C., preferably 15-30° C., more preferably 18-24° C., and at a pressure of 1 atm, or
preferably wherein the galacto-oligosacharides comprised in the fermented milk-based product, such as yogurt or yogurt product, are stable for 1 day to 250 days, more preferably for 1 day to 200 days, even more preferably for 1 day to 180 days, at a temperature of 15-37° C., preferably 15-30° C., more preferably 18-24° C., and at a pressure of 1 atm.

The method may optionally comprise a further step of storing the fermented milk-based product comprising galacto-oligosaccharides at a temperature of 15-37° C., preferably 15-30° C., more preferably 18-24° C., and at a pressure of 1 atm.

The method may optionally be carried out by using a milk-based substrate comprising at least 1% W_{lactose}/W_{milk-based substrate}, preferably 1-60% W_{lactose}/W_{milk-based substrate}, more preferably 2-50% W_{lactose}/W_{milk-based substrate}, even more preferably 3-40% W_{lactose}/W_{milk-based substrate}.

The method may also be optionally carried out using as milk-based substrate milk selected from: cow milk, sheep milk, goat milk, buffalo milk, camel milk, pasteurized milk, raw milk, filtered milk, or combinations thereof.

Optionally, the step of treating the milk-based substrate with a beta-galactosidase enzyme to generate galacto-oligosaccharides may be carried out for 1-24 hours, preferably 3-22 hours, more preferably 5-18 hours and/or at a temperature of 15-60° C., preferably 25-45° C., more preferably at 43° C.

Optionally, the step of treating the milk-based substrate with a beta-galactosidase enzyme is carried out with at least 0.1 g/L_{milk-based substrate} of beta-galactosidase enzyme or at most 10 g/L_{milk-based substrate} of beta-galactosidase enzyme or with less than 10 g/L_{milk-based substrate} of beta-galactosidase enzyme, preferably with 0.1-10 g/L_{milk-based substrate} of beta-galactosidase enzyme or 0.1-9 g/L_{milk-based substrate} of beta-galactosidase enzyme or 0.1-8 g/L_{milk-based substrate} of beta-galactosidase enzyme, more preferably with 0.2-7 g/L_{milk-based substrate} of beta-galactosidase enzyme or 0.3-8 g/L_{milk-based substrate} of beta-galactosidase enzyme, even more preferably with 0.3-5 g/L_{milk-based substrate} of beta-galactosidase enzyme or 0.3-3 g/L_{milk-based substrate} of beta-galactosidase enzyme or 0.5-1 g/L_{milk-based substrate} of beta-galactosidase enzyme. Alternatively, in the context of this invention, 1 g (including the tested dosages range of 0.1 g to 10 g) of enzyme dosage per liter of milk or per liter of milk-based substrate may refer or refers to 1 g of a commercial enzyme product, such as Saphera Fiber or Nurica, having a defined activity (as per certificate of analysis by the supplier) in 3000-3300 LAU-C/g or 600-650 U/g applied to 1 L of milk, respectively. Despite having different activity units for Saphera Fiber and Nurica both test enzymes were applied in equal weights in grams per liter of milk or milk-based substrate.

Additionally, “g/L_{milk-based substrate} of beta-galactosidase enzyme” and “g_{beta-galactosidase enzyme}/L_{milk-based substrate}” have the meaning and are, in the entire disclosure interchangeable.

Optionally, the method is carried out using a Bifidobacterium beta-galactosidase enzyme, preferably wherein the beta-galactosidase enzyme is a Bifidobacterium bifidum beta-galactosidase, more preferably wherein the beta-galactosidase enzyme has at least 80% or at least 85% or at least 90% or at least 95% or at least 96% or at least 97% or at least 98% or at least 99% or 100% sequence identity to SEQ ID NOs 1, 2, 3, 4, 5, 6, 7, 8, 9, 10, 11, 12, 13, 14, or 15 or a sequence, even more preferably wherein the sequence is SEQ ID NO 1 or SEQ ID NO 7.

The method may be carried out by using a starter culture for achieving the fermentation of the milk-based substrate or milk-based substrate comprising galacto-oligosaccharides.

The step of inactivating the beta-galactosidase enzyme may be carried out at a pH below 5, such as for example at a pH between 3.4 to 4.5, preferably between 3.6 to 4.4, more preferably 3.8 to 4.3, and from 70° C. to less than 95° C., preferably from 70° C. to 90° C. or from 70° C. to 85° C. or preferably from 72° C. to 80° C., more preferably from 70° C. to 75° C. Further, the step of inactivating the beta-galactosidase enzyme may also be carried out for about 5 seconds to 30 minutes, or for about 10 seconds to 20 minutes, or for about 15 seconds to 15 minutes, or for about 20 seconds to about 5 minutes, or for about 5 seconds to about 20 seconds or for about 5 seconds to 10 seconds or for about 10 to 20 seconds. Preferably, the step of inactivating the beta-galactosidase enzyme may be carried from 70° C. to 75° C. for 5 seconds to 60 seconds, preferably from 70° C. to 75° C. for 10 seconds to 40 seconds, more preferably from 70° C. to 75° C. for 15 seconds to 20 seconds, more preferably 72° C. for 20 seconds or 75° C. for 20 seconds. Finally and preferably, the step of inactivating the beta-galactosidase may be carried out from at 70° C. to 75° C. for 10 seconds to 60 seconds and at a pH below 5, preferably from 70° C. to 75° C. for 20 seconds to 60 seconds and at a pH below 5, more preferably from 72° C. for 20 seconds and at a pH of 4.3 or from 75° C. for 20 seconds and at a pH of 4.3.

Optionally, the method now disclosed may be a method for producing a fermented milk-based product, wherein said product is yoghurt, buttermilk, creme fraiche, quark, fromage frais, yakult or skyr; or wherein the fermented milk product is a stirred fermented milk product or drinking fermented milk product, preferably wherein the stirred fermented milk product is a stirred yogurt or the drinking fermented milk product is drinking yogurt, more preferably wherein the stirred yogurt is a post-pasteurization stirred yogurt or the drinking yogurt is an post-pasteurization drinking yogurt.

This invention also relates to a fermented milk-based product comprising galacto-oligosaccharides obtained by the method therein disclosed, in particular yogurt or yogurt product comprising galacto-oligosaccharides obtained by the method therein disclosed.

### Fermented Milk-Based Product, Preferably Yogurt or Yogurt Product, or More Preferably Ambient Yogurt, Comprising Galacto-Oligosaccharides

This invention also relates to a fermented milk-based product comprising galacto-oligosaccharides, in particular a yogurt or an ambient yogurt or a yogurt product or a ambient yogurt product, wherein the pH of the fermented milk-based product is below 5, preferably between 3.4 to 4.5, more preferably between 3.6 to 4.4, even more preferably 3.8 to 4.3; and wherein the fermented milk-based product is stored at a temperature of 15-37° C., preferably 15-30° C., more preferably 18-24° C. and at a pressure of 1 atm, preferably further comprising an inactivated beta-galactosidase enzyme, preferably wherein the inactivated beta-galactosidase enzyme is a Bifidobacterium beta-galactosidase enzyme ory wherein the inactivated beta-galactosidase enzyme is a Bifidobacterium bifidum beta-galactosidase, more preferably wherein the inactivated beta-galactosidase enzyme has at least 80% or at least 85% or at least 90% or at least 95% or at least 96% or at least 97% or at least 98% or at least 99% or 100% sequence identity to SEQ ID NOs 1, 2, 3, 4, 5, 6, 7, 8, 9, 10, 11, 12, 13, 14, or 15 or a sequence, even more preferably wherein the sequence is SEQ ID NO 1 or SEQ ID NO 7.

Preferably, the fermented milk-based product, in particular a yogurt or an ambient yogurt or a yogurt product or a ambient yogurt product, may comprise at least 0.5% W_{galacto-oligosaccharides}/W_{fermented milk-based product}, more preferably 0.5-4% W_{galacto-oligosaccharides}/W_{fermented milk-based product}, even more preferably 1-3% W_{galacto-oligosaccharides}/W_{fermented milk-based product} or 1.5-2.5% W_{galacto-oligosaccharides}/W_{fermented milk-based product}. If the fermented milk-based product is a yogurt then the units are adjusted for the product. As an example, yogurt may comprise at least 0.5% W_{galacto-oligosaccharides}/W_{yogurt}, more preferably 0.5-4% W_{galacto-oligosaccharides}/W_{yogurt}, even more preferably 1-3% W_{galacto-oligosaccharides}/W_{yogurt} or 1.5-2.5% W_{galacto-oligosaccharides}/W_{yogurt}.

Preferably, the galacto-oligosaccharides comprised in the fermented milk-based product, in particular a yogurt or an ambient yogurt or a yogurt product or a ambient yogurt product, may have a DP of more than 2 or at least 3; more preferably the galacto-oligosaccharides comprise or include lactosucrose.

Preferably, the fermented milk-based product, in particular a yogurt or an ambient yogurt or a yogurt product or a ambient yogurt product, may comprise at least 0.1% W_{galacto-oligosaccharides with DP>2}/W_{fermented milk-based product}, more preferably 0.2-3% W_{galacto-oligosaccharides with DP>2}/W_{fermented milk-based product}, even more preferably 0.3-2% W_{galacto-oligosaccharides with DP>2}/W_{fermented milk-based product} or 0.4-1% W_{galacto-oligosaccharides with DP>2}/W_{fermented milk-based product}. If the fermented milk-based product is a yogurt then the units are adjusted for the product. As an example, yogurt may comprise 0.1% W_{galacto-oligosaccharides with DP>2}/W_{yogurt}, more preferably 0.2-3% W_{galacto-oligosaccharides with DP>2}/W_{yogurt}, even more preferably 0.3-2% W_{galacto-oligosaccharides with DP>2}/W_{yogurt} or 0.4-1% W_{galacto-oligosaccharides with DP>2}/W_{yogurt}.

Preferably, the fermented milk-based product comprising galacto-oligosaccharides may be a yoghurt, a yogurt product, buttermilk, creme fraiche, quark, fromage frais, yakult or skyr. The fermented milk product may be a stirred fermented milk product, preferably the stirred fermented milk product may be a stirred yogurt or a stirred yogurt product, more preferably the stirred yogurt may be a post-pasteurization stirred yogurt or a post-pasteurization stirred yogurt product; or the fermented milk product may be drinking fermented milk product, preferably the drinking fermented milk product may be a drinking yogurt or a drinking yogurt product, more preferably drinking yogurt may be is a post-pasteurization drinking yogurt or a post-pasteurization drinking yogurt product.

Preferably, the galacto-oligosacharides comprised in the fermented milk-based product may be stable for at least 5 days at a temperature of 15-37° C., preferably 15-30° C., more preferably 18-24° C., and at a pressure of 1 atm, preferably for at least 10 days, more preferably for at least 15 days, even more preferably for at least 21 days.

### Method for Inactivating a Beta-Galactosidase Enzyme

This invention also concerns a method for inactivating a beta-galactosidase enzyme comprising the step of submitting the beta-galactosidase enzyme to a temperature below 95° C. at a pH below 5, preferably comprising the step of submitting the beta-galactosidase enzyme to a temperature from 70° C. to 85° C. and a pH below 5.

Optionally, the method for inactivating a beta-galactosidase enzyme may be carried out at a temperature from 70° C. to less than 95° C., preferably from 70° C. to 90° C., preferably from 70° C. to 85° C. or more preferably from 72° C. to 80° C., even more preferably from 70° C. to 75° C.

Optionally, the method for inactivating a beta-galactosidase enzyme may be carried out at a pH between 3.4 to 4.5, preferably between 3.6 to 4.4, more preferably 3.8 to 4.3.

Optionally, the method for inactivating a beta-galactosidase enzyme may be carried out at a temperature from 70° C. to less than 95° C., preferably from 70° C. to 90° C., preferably from 70° C. to 85° C. or more preferably from 72° C. to 80° C., even more preferably from 70° C. to 75° C., and at a pH between 3.4 to 4.5, preferably between 3.6 to 4.4, more preferably 3.8 to 4.3.

Optionally, the method for inactivating a beta-galactosidase enzyme may be carried out for about 5 seconds to 30 minutes, or for about 10 seconds to 20 minutes, or for about 15 seconds to 15 minutes, or for about 20 seconds to about 5 minutes. Preferably, the method may be carried out for about 5 seconds to about 20 seconds or for about 5 seconds to 10 seconds or for about 10 to 20 seconds.

Optionally, the method relates to the inactivation of a Bifidobacterium beta-galactosidase enzyme, preferably wherein the beta-galactosidase enzyme is a Bifidobacterium bifidum beta-galactosidase, more preferably wherein the beta-galactosidase enzyme has at least 80%, at least 85%, at least 90%, at least 95%, at least 96%, at least 97%, at least 98%, at least 99% or 100% sequence identity to SEQ ID NOs 1, 2, 3, 4, 5, 6, 7, 8, 9, 10, 11, 12, 13, 14, or 15 or a sequence, even more preferably wherein the sequence is SEQ ID NO 1 or SEQ ID NO 7.

### Definitions

In connection with the present invention the following definitions apply.

GOS generally comprise a chain of galactose units that arise through consecutive trans-galactosylation reactions, with a terminal glucose unit or a terminal fructose unit if sucrose is present in the reaction mixture. The DP of GOS can vary quite markedly, depending mainly on the type of the beta-galactosidase used and the conversion degree of lactose. “Degree of polymerization” or “DP” of GOS refers to the total number of sugar monomer units that are part of the particular a oligosaccharide. For example, a DP of 2 corresponds to a GOS having 1 galactose moiety and 1 glucose moiety or a fructose moiety; a DP of 3 corresponds to a GOS having 2 galactose moieties and 1 glucose moiety or a fructose moiety; a DP of 4 corresponds to a GOS having 3 galactose moieties and 1 glucose moiety or a fructose moiety, and so on.

“Total GOS” is herein defined as the total amount or concentration of GOS generated by the beta-galactosidase. Total GOS includes oligosaccharides with a DP of 2 or more. Examples of GOS with a DP of 2 include, for example but not limited to, allolactose, lactulose, 1,3-b-Gal-Glc. Lactose having a DP of 2 is excluded from the definition of total GOS formed used herein.

“GOS fiber” is herein defined as non-digestible GOS having a DP above 2 (>DP2), meaning GOS having a DP of 3 or above rates with 3 or more monomeric units. Examples of GOS with a DP>2 include, for example but not limited to, lactosucrose, 6′-galactosyllactose, 4-gal-lact, 3-gal-lact. In the context of the present invention, GOS with DP>2 or GOS/fiber or dietary fiber are synonyms and herein interchangeable.

“Room temperature” or “ambient temperature” is herein defined as the range of air temperatures between 15-37° C., preferably 15-30° C., more preferably 18-24° C., either range at a pressure of 1 atm. The expressions “room temperature” and “ambient temperature” are herein interchangeable.

“Ambient storage food product” or “ambient food product” means a fermented milk product, which is suitable for ambient storage for a period of time, wherein ambient storage is herein defined as range of air temperatures between 15-37° C., preferably 15-30° C., more preferably 18-24° C., either range at a pressure of 1 atm that equals 101.325 kPa.

“Heat treated fermented milk product” means a fermented milk product, which has been subjected to heat treatment.

“Milk” is to be understood as the lacteal secretion obtained by milking of any mammal, such as cows, sheep, goats, buffaloes or camels. In a preferred embodiment, the milk is cow's milk. The term milk also includes protein/fat solutions made of plant materials, e.g. soy milk, provided lactose is present.

“Milk substrate” or “milk-based substrate” may be any raw and/or processed milk material that can be subjected to fermentation according to the method of the invention. Thus, useful milk substrates include, but are not limited to, solutions/-suspensions of any milk or milk like products comprising lactose, preferably comprising at least 0.002% (0.002 g/100 ml) of lactose, such as whole or low-fat milk, skim milk, buttermilk, reconstituted milk powder, condensed milk, dried milk, whey, whey permeate, whey protein concentrate, acid whey, cream, fermented milk products, such as yogurt or cheese. The “milk substrate” or “milk-based substrate” may originate from any mammal, e.g. being substantially pure mammalian milk, or reconstituted milk powder. Typically the term “milk substrate” or “milk-based substrate” refers to a raw or processed milk material that is processed further in order to produce a dairy product. Prior to fermentation, the “milk substrate” may be homogenized and pasteurized according to methods known in the art.

“Homogenizing” as used herein means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices.

“Pasteurization” as used herein means treatment of the milk substrate to reduce or eliminate the presence of live organisms, such as microorganisms, in a milk-based substrate. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

“Fermentation” as used herein means the conversion of carbohydrates into alcohols or acids through the action of a microorganism. Preferably, fermentation in the methods of the invention comprises conversion of lactose to lactic acid. To ferment the milk-based substrate a starter culture is added.

Fermentation processes to be used in production of dairy products are well known and the person of skill in the art will know how to select suitable process conditions, such as temperature, oxygen, amount and characteristics of microorganism(s) and process time. Fermentation conditions are selected so as to support the achievement of the present invention, i.e. to obtain a dairy product in solid form (such as a cheese) or liquid form (such as a fermented milk product).

“Fermented dairy product” or “fermented milk product” as used herein is to be understood as any dairy product wherein any type of fermentation forms part of the production process. Examples of fermented dairy products are products like yoghurt, buttermilk, creme fraiche, quark and fromage frais. A fermented dairy product may be produced by or include steps of any method known in the art.

“Starter” or “starter culture” as used in the present context refers to a culture of one or more food-grade microorganisms in particular lactic acid bacteria, which are responsible for the acidification of the milk base. Starter cultures may be fresh, frozen or freeze-dried. It is within the skills of ordinary practitioners to determine the starter culture and amounts to be used.

“Dairy product” as used herein may be any food product wherein one of the major constituents is milk-based. Usually the major constituent is milk-based and in some embodiments, the major constituent is a milk-based substrate which has been treated with an enzyme having beta-galactosidase activity according to a method of the present invention.

A dairy product according to the invention may be, e.g., skim milk, low fat milk, whole milk, cream, UHT milk, milk having an extended shelf life, a fermented milk product, cheese, yoghurt, butter, dairy spread, butter milk, acidified milk drink, sour cream, whey-based drink, ice cream, condensed milk, dulce de leche or a flavored milk drink.

A dairy product may additionally comprise non-milk components, e.g. vegetable components such as, e.g., vegetable oil, vegetable protein, and/or vegetable carbohydrates. Dairy products may also comprise further additives such as, e.g., enzymes, flavoring agents, microbial cultures such as probiotic cultures, salts, sweeteners, sugars, acids, fruit, fruit prep fruit juices, or any other component known in the art as a component of, or additive to, a dairy product.

“Sequence identity” for amino acids as used herein refers to the sequence identity calculated as (n_{ref}−n_{dif})·100/n_{ref}, wherein n_{dif} is the total number of non-identical residues in the two sequences when aligned and wherein n_{ref} is the number of residues in one of the sequences.

In some embodiments the sequence identity is determined by conventional methods, e.g., Smith and Waterman, 1981, Adv. Appl. Math. 2:482, by the search for similarity method of Pearson & Lipman, 1988, Proc. Natl. Acad. Sci. USA 85:2444, using the CLUSTAL W algorithm of Thompson et al., 1994, Nucleic Acids Res 22:467380, by computerized implementations of these algorithms (GAP, BESTFIT, FASTA, and TFASTA in the Wisconsin Genetics Software Package, Genetics Computer Group). The BLAST algorithm (Altschul et al., 1990, Mol. Biol. 215:403-10) for which software may be obtained through the National Center for Biotechnology Information www.ncbi.nlm.nih.gov/) may also be used. When using any of the aforementioned algorithms, the default parameters for “Window” length, gap penalty, etc., are used.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### DETAILED DESCRIPTION OF THE INVENTION

The invention relates to a method for producing a fermented milk-based product, preferably yogurt or yogurt product, or more preferably ambient yogurt, comprising GOS formed in-situ and low levels of lactose, comprising a step of inactivating the beta-galactosidase enzyme used for the in-situ GOS formation. This inactivating step is carried out by a combination of a temperature below 95° C. and at a pH below 5. The inactivation of the beta-galactosidase enzyme after the GOS formation takes place is responsible for maintaining GOS are stable over time (as GOS can also be a substrate for the beta-galactosidase) and wherein the fermented milk-based product can be stored at room temperature without perish.

In the context of the present invention, any Bifidobacterium derived beta-galactosidase or Bifidobacterium beta-galactosidase may be used, in particular a Bifidobacterium bifidum derived beta-galactosidase or Bifidobacterium bifidum beta-galactosidase. Several prior art documents disclosed examples of Bifidobacterium derived beta-galactosidase and/or Bifidobacterium bifidum derived beta-galactosidase.

For example, the sequences disclosed in WO 2013/182686 or WO 2020/117548 can be herein used, as well as methods to obtain and/or treat said sequences, such as after expressing said sequences. In particular SEQ ID Nos 1 to 6 of WO 2013/182686 correspond to SEQ ID Nos 1 to 6, respectively of the present invention. In particular SEQ ID Nos 1 to 5 of WO 2020/117548 correspond to SEQ ID Nos 1 to 5, respectively, of the present invention. Preferably SEQ ID No 1 of WO 2013/182686 or SEQ ID No 1 of WO 2020/117548 is used or SEQ ID No 1 of the present invention is used. Alternatively, Dupont™ Danisco®) Nurica™ may be used. In the context of the present invention, SEQ ID No 1 and Nurica™ are used or may be used interchangeably.

In alternative or in addition, the sequences of WO 2018/210820 can also be herein used, as well as methods to obtain and/or treat said sequences, such as after expressing said sequences and/or submitting said sequences to a glycosylation step as described in Example 5 of WO 2018/210820. In particular SEQ ID Nos 1 to 12 of WO 2018/210820 correspond to SEQ ID Nos 7 to 18, respectively, of the present invention. Preferably SEQ ID No 1 of WO 2018/210820 corresponds to SEQ ID No 7 of the present invention, wherein the sequence has been submitted to a glycosylation or glycation step (glycosylation and glycation are used interchangeably) under the conditions explained in of WO 2018/210820. Alternatively, Saphera®) Fiber available from Novozymes A/S may be used. In the context of the present invention, SEQ ID No: 7 glycosylated and Saphera®) Fiber are used or may be used interchangeably.

In the present invention, the method for quantification of monosaccharides, dissacharides and GOS may be carried out as follows:
GOS, as well as mono- and disaccharides were analyzed by high-performance anion-exchange chromatography with pulsed amperometric detection (HPAE-PAD), on a Dionex ICS-5000, ICS-6000 or Integrion system (Thermo Fischer Scientific, Waltham, MA, USA). The systems were equipped with a Dionex™ CarboPac™ SA10 column (4 mm×250 mm, 6 μM), and a EGC KOH Eluent Generator Cartridge, that was used to generate a gradient starting with 1 mM KOH for 10 min, which was linear increased to 20 mM over 20, and to 100 mM over further 20 min, where it was held for 20 min, before reverting to 1 mM in 0.1 min, keeping this for 20 min. Flow rate was 1.0 ml/min and column temperature 30° C. Water (MQ) was purified from a Milli-Q water purifier (Millipore, Burlington, MA, USA), to 18.2 MΩ×cm and a TOC<5 ppb. To avoid up-concentration of milk impurities in the system, the CR-CT trap was continuously feed with 0.5 ml/min fresh MQ from an extra AXP pump (Thermo Fischer Scientific), while an additional AXP pump feeding 0.5 ml/min 300 mM NaOH post column to boost detector signal.Fucose was used as internal standard, while, fructose, galactose, glucose, lactose, tagatose, trehalose (Sigma-Aldrich Chemie Gmbh, Munich, Germany), allo-lactose, 1,3-β-D-Galactosyl-D-glucose, 6′-Galactosyllactose, 4′-Galactosyllactose, and 3′-Galactosyllactose (Carbosynth Limited, Compton, UK) were analyzed for external standard quantification, based on peak areas. Bimuno Bi2Muno Prebiotic Food Supplement Sachets (Clasado Limited, Shinfield, Reading, UK) with GOS were diluted in MQ used as quality control samples.
Milk and yoghurt samples (1.0 g) were quenched with 2 ml 96% ethanol and frozen. After thawing they were diluted with 2 ml MQ and a subsample centrifuged at 21000 g for 10 min, the supernatant was then further diluted to 500 times with MQ containing 0.02% (w/v) sodium azide (Sigma-Aldrich), and 3 μl sub-samples injected in the HPAE-PAD system.

There are, however, alternative methods for quantifying monosaccharides, disaccharides and GOS, which have been, for example, disclosed in WO2013/182686, WO2015/132402, WO2020/117548 or WO2018/210820.

### EXAMPLES

### Example 1—Preparation of Pasteurized Milk

A beta-galactosidase enzyme was added to semi-skimmed milk with 4.7% lactose, 1.5% fat, 3.8% protein (estimated), as shown in Table 1, kept at 5° C. for 24h, and then heat treated at 72° C. for 20s or 72° C. for 40s. In this example, the beta-galactosidase used was Saphera®) Fiber. Alternatively, any other beta-galactosidase as the ones herein disclosed may be used as well. Identical results are expected.

Samples were obtained after heat treatment as follows: day 0, day 2 at 5° C. and after 1 week at 5° C. and the GOS stability levels were quantified as described above and are shown in

### Table 1.

TABLE 1
GOS stability levels at indicated days
of storage at 5° C. in pasteurized milk.

| | | Total GOS (mg/g) | | | | | |
|---|---|---|---|---|---|---|---|
| | Dosage of | 20 sec | | | 40 sec | | |
| | enzyme | Day | Day | Week | Day | Day | Week |
| Sample | (g/L) | 0 | 2 | 1 | 0 | 2 | 1 |
| A | 0 | 0.00 | 0.00 | 0.00 | 0.00 | 0.00 | 0.00 |
| B | 3.0 | 10.2 | 7.6 | 7.3 | 12.7 | 11.3 | 9.3 |
| C | 4.0 | 7.9 | 5.6 | 4.6 | 11.5 | 11.4 | 8.9 |
| D | 5.0 | 6.7 | 4.5 | 4.0 | 11.4 | 8.8 | 8.4 |

As seen from Table 1, the level of GOS decreased over time independently of the pasteurization conditions (72° C. for 20 sec or 72° C. for 40 sec), indicating a heat treatment of 72° C. for 20s or 72° C. for 40s does not contribute for maintaining the levels of in-situ GOS stable over time, as the beta-galactosidase enzyme used is not (fully) inactivated by the heat treatment performed.

### Example 2—Preparation of Cold Yogurt

Starter culture (culture of lactic acid bacteria) and beta-galactosidase were added to a milk base formulated to have a protein level of 4.0% and a fat level of 1.0% by using semi-skimmed milk, full fat milk and SMP (Skimmed milk powder). The inoculation level of the stater culture was 0.2 U/L, while the beta-galactosidase was added as shown in Table 2. In this example, the beta-galactosidase used was Saphera® Fiber. Alternatively, any other beta-galactosidase as the ones herein disclosed may be used as well. Identical results are expected.

The starter culture can be any culture developed for fermented milk applications or thermophilic fermented milk applications. These cultures are well known in the art. As an example, a culture can be YoFlex® starter culture type, commercialized by Chr. Hansen A/S, for example the starter culture may be YoFlex® Premium 2.0 available from Chr. Hansen A/S; however, there are many more examples well known to the skilled person as alternative starter cultures.

Samples were obtained at day 0 and day 35. The pH was determined, as well as the GOS stability levels, which were quantified as described above and are shown in Table 2.

TABLE 2
GOS stability levels at indicated days
of storage at 5° C. in cold yogurt.

Total GOS (mg/g)

| | Dosage of enzyme | Day 0 | Day 35 |
|---|---|---|---|
| Sample | (g/L) | pH 4.55 | pH of 4.29 to 4.35 |
| E | 0 | 0.6 | 0.9 |
| F | 0.5 | 7.0 | 2.9 |
| G | 1.0 | 6.6 | 1.5 |
| H | 2.0 | 3.0 | 0.1 |
| I | 3.0 | 1.5 | 0.1 |

Table 2 shows the total GOS stability levels, decreased over, indicating that a pH of 4.29 to 4.35 does not contribute for maintaining the levels of in-situ GOS stable over time, as the beta-galactosidase enzyme used is not inactivated by this pH range.

In conclusion, Examples 1 and 2 show that pasteurization of neutral milk by 75° C., 20s (Example 1), as well as low pH, such a pH of ≤4.30 (Example 2) in fermented milk products is not sufficient to (fully) inactivate the beta-galactosidase enzyme used to reduce lactose levels in a milk-based product while simultaneously being responsible for the in-situ GOS formation.

### Example 3—Preparation of Ambient Yogurt and Stability of GOS at 21 Days

Yogurt milk was formulated to a typical formulation in the ambient yogurt category. The milk-base substrate consisted of commercial fresh milk (3.5% fat), water, whey protein concentrate, 7% sucrose, 1.5% modified starch, 0.12% LM-Pectin, 0.03% gellan gum, with a calculated lactose level of 3.6%. After hydration of ingredients at 5° C. overnight, the milk was homogenized (150/50 bar at 60° C.) and pasteurized, in particular at 95° C. for 300 seconds. The milk was cooled to 43° C. Starter culture (culture of lactic acid bacteria) and beta-galactosidase were added simultaneously to the milk once it reached a temperature of 43° C. The inoculation level of the starter culture was 0.2 U/L, independently of the starter culture used, while the beta-galactosidase was added as shown in Table 3.

TABLE 3
Cultures, enzymes and dosages used in Example 3.

| | | | Dosage of enzyme |
|---|---|---|---|
| Sample | Culture | Enzyme | (g/L) |
| 1 | FD-DVS ® YF-L904 | Reference | 0 |
| 2 | | Saphera ® Fiber | 0.5 |
| 3 | | | 1.0 |
| 4 | F-DVS ® YF-L909 | Reference | 0 |
| 5 | | Saphera ® Fiber | 0.5 |
| 6 | | Nurica ™ | 0.5 |

The starter culture can be any culture developed for fermented milk applications or thermophilic fermented milk applications. These cultures are well known in the art. As an example, a culture can be YoFlex® starter culture type FD-DVS® YF-L904 containing the two strains Streptococcus thermophilus and Lactobacillus delbrueckii spp. bulgaricus or type F-DVS® YF-L909, both commercialized by Chr. Hansen A/S; however, there are many more examples well known to the skilled person as alternative starter cultures.

Following the standard process for ambient yogurt, fermentation was done to pH 4.30 at 43° C., prior to final heat treatment at 75° C., 20s, cooling to 25° C. and packaging to sterile cups. Samples were stored at room temperature (or ambient temperature). This process is known to inactivate the starter culture, and together with the low pH and sterile packaging ensuring the shelf-life of 6-9 months.

Sampling for carbohydrates, including GOS, for quantification was done on day 0 and day 21. The quantification of residual lactose, galactose and GOS (total GOS and GOS DP>2) was performed as explained above. In particular, one of the GOS formed may be lactosucrose.

The comparison of the stability levels of total GOS and fiber (GOS with >DP2), as well as remaining lactose levels and galactose levels, in ambient yogurt samples, is provided in Table 4.

TABLE 4
GOS stability levels at indicated days of storage at ambient
temperature (T = 25° C.) in ambient yogurt.

| | Total GOS | | GOS DP > 2, | | Residual lactose | | Galactose | |
|---|---|---|---|---|---|---|---|---|
| | (mg/g) | | Fiber (mg/g) | | (mg/g) | | (mg/g) | |
| Sample | Day 0 | Day 21 | Day 0 | Day 21 | Day 0 | Day 21 | Day 0 | Day 21 |
| 1 | 0.00 | 0.00 | 0.00 | 0.00 | 23.7 | 22.9 | 4.3 | 4.2 |
| 2 | 17.5 | 16.5 | 15.7 | 14.6 | 7.3 | 6.9 | 4.6 | 4.5 |
| 3 | 17.2 | 16.8 | 14.7 | 14.3 | 3.0 | 3.1 | 5.8 | 5.7 |
| 4 | 0.00 | 0.00 | 0.00 | 0.00 | 25.7 | 25.0 | 4.2 | 4.1 |
| 5 | 17.8 | 17.1 | 16.2 | 15.4 | 9.6 | 9.2 | 4.3 | 4.3 |
| 6 | 19.4 | 18.7 | 17.6 | 17.0 | 8.4 | 7.9 | 4.0 | 3.9 |

Table 4 discloses the following:
the measured level of total GOS, GOS having a DP>2, residual lactose and galactose remain stable for at least 21 days of storage at 25° C., indicating that the beta-galactosidases used have been inactivated by heat treatment performed at low pH-see data for samples 2,3, 5 and 6;
either enzyme used contributes similarly to the amount of total GOS, fiber (DP>2) and galactose formed remaining in the samples after 21 days of storage-see samples 2, 5 and 6;
samples 2-3 and 5-6 have significantly lower levels of residual lactose than the reference samples (samples 1 and 4), showing that the method herein disclosed reduces the lactose in the milk-based substrate leading to a low lactose milk-based product, while simultaneously generating GOS which are stable for at least 21 days at 25° C.;
the residual lactose levels is lowest when 1.0 g/L of enzyme is used-sample 3 versus remaining samples 2 and 5-6.

Further, GOS stability levels are expected to be maintained even after longer periods of time, such as for 90 days or 180 days, as a result of the enzyme inactivation.

### Example 4—Preparation of Ambient Yogurt and Stability of GOS at 90 Days

Ambient yogurt milk was prepared as explained in Example 3. The dosage of beta-galactosidase used was as shown in Table 5. In this example, the beta-galactosidase used was Saphera® Fiber. Alternatively, any other beta-galactosidase as the ones herein disclosed may be used as well. Identical results are expected.

TABLE 5
Cultures, enzymes and dosages used in Example 4.

| | | Dosage of enzyme |
|---|---|---|
| Sample | Culture | (g/L) |
| 7 | FD-DVS ® YF-L904 | 0 |
| 8 | | 0.5 |
| 9 | | 1.0 |
| 10 | F-DVS ® YF-L909 | 0 |
| 11 | | 0.5 |

Sampling for total GOS quantification was done on days 0 and 90. This quantification was performed as explained above. The stability levels of total GOS in ambient yogurt samples over time is provided in Table 6.

TABLE 6
GOS stability levels at indicated days of storage at ambient
temperature (T = 25° C.) in ambient yogurt.

Total GOS
(mg/g)

| Sample | Day 0 | Day 90 |
|---|---|---|
| 7 | 0.6 | 0.6 |
| 8 | 13.9 | 15.7 |
| 9 | 19.8 | 16.4 |
| 10 | 0.7 | 0.7 |
| 11 | 15.1 | 14.9 |

### Example 5—Preparation of Ambient Yogurt and Stability of GOS at 180 Days

Ambient yogurt milk was prepared as explained in Example 3. The dosage of beta-galactosidase used was as shown in Table 7. In Example 5, the beta-galactosidase used was Saphera® Fiber. Alternatively, any other beta-galactosidase as the ones herein disclosed may be used as well. Identical results are expected.

TABLE 7
Cultures, enzymes and dosages used in Example 4.

| | | Dosage of enzyme |
|---|---|---|
| Sample | Culture | (g/L) |
| 12 | FD-DVS ® YF-L904 | 0 |
| 13 | | 0.5 |
| 14 | | 1.0 |
| 15 | F-DVS ® YF-L909 | 0 |
| 16 | | 0.5 |

Sampling for total GOS quantification was done on days 0 and 180. This quantification was performed as explained above. The stability levels of total GOS in ambient yogurt samples over time is provided in Table 8.

TABLE 8
GOS stability levels at indicated days of storage at ambient
temperature (T = 25° C.) in ambient yogurt.

Total GOS
(mg/g)

| Sample | Day 0 | Day 180 |
|---|---|---|
| 12 | 0.5 | 0.7 |
| 13 | 18.3 | 18.1 |
| 14 | 23.9 | 20.6 |
| 15 | 0.6 | 0.8 |
| 16 | 17.0 | 16.9 |

### Examples 4 and 5 Show that GOS (Total GOS) is Stable

1) in ambient yogurt, when the beta-galactosidase is inactivated by performing heat treatment from 70° C. to 85° C. at a pH below 5;
2) at room temperature; and
3) for at least 21 days, preferably at least 90 days, more preferably at least 180 days after their in situ production.

Identical results to the ones herein disclosed, in particular regarding the GOS stability, are expected for SEQ ID Nos: 1-18, Nurica or any other beta-galactosidase enzyme.

### Example 6—Preparation of Ambient Yogurt and Stability of GOS at 21, 90 and 180 Days

Ambient yogurt milk was prepared as explained in Example 3. The dosage of beta-galactosidase used was as shown in Table 9. In this example the beta-galactosidase used was Saphera® Fiber. Alternatively, any other beta-galactosidase as the ones herein disclosed may be used as well. Identical results are expected.

TABLE 9
Cultures, enzymes and dosages used in Example 6.

| | | Dosage of enzyme |
|---|---|---|
| Sample | Culture | (g/L) |
| 17 | FD-DVS ® YF-L904 | 0.1 |
| 18 | | 0.3 |
| 19 | | 3 |
| 20 | | 5 |
| 21 | | 8 |
| 22 | | 10 |

Sampling of carbohydrates, including GOS, for quantification was done on days 0, 21, 90 and 180. The quantification of GOS (total GOS and GOS DP>2) was performed as explained above. In particular, one of the GOS formed may be lactosucrose.

The comparison of the stability levels of total GOS and fiber (GOS with >DP2) in ambient yogurt samples, is provided in Table 10.

TABLE 10
GOS stability levels at indicated days of storage at ambient
temperature (T = 25° C.) in ambient yogurt.

| | Total GOS (mg/g) | | | | GOS DP > 2, Fiber (mg/g) | | | |
|---|---|---|---|---|---|---|---|---|
| Sample | Day 0 | Day 21 | Day 90 | Day 180 | Day 0 | Day 21 | Day 90 | Day 180 |
| 17 | 8.0 | 8.2 | 9.1 | 8.9 | 6.9 | 7.1 | 7.6 | 7.2 |
| 18 | 12.2 | 12.6 | 12.9 | 12.8 | 9.8 | 10.1 | 10.1 | 9.8 |
| 19 | 27.9 | 25.7 | 27.1 | 29.5 | 18.7 | 17.9 | 18.2 | 19.4 |
| 20 | 20.0 | 20.5 | 18.9 | 19.5 | 14.6 | 15.3 | 13.8 | 14.0 |
| 21 | 10.6 | 11.1 | 9.4 | 10.2 | 8.0 | 8.5 | 6.7 | 7.6 |
| 22 | 5.1 | 7.2 | 6.0 | 6.1 | 3.7 | 5.3 | 4.5 | 4.5 |

Table 10 discloses that the measured level of total GOS and GOS having a DP>2 remain stable for at least 180 days of storage at 25° C., independently of the dosage of enzyme used and indicating that the beta-galactosidase used has been inactivated by heat treatment performed at low pH.

In conclusion, it was unknown that a combination of heat treatment at low pH would lead to the inactivation of the beta-galactosidase enzyme used for the in situ GOS formation and to the maintenance of stable GOS levels over time. In particular, the present invention shows that GOS, either total GOS or GOS having a DP>2, are stable:
1) in a fermented milk-based product, preferably in a low-lactose fermented milk-based product, more preferably in ambient yogurt, when the beta-galactosidase is inactivated by performing heat treatment below 95° C. at a pH below 5, in particular from 70° C. to 85° C. at a pH below 5;
2) at room temperature; and
3) for at least 21 days, preferably 90 days, more preferably 180 days after their in situ production.

### REFERENCES

WO 2013/182686, WO 2015/132402, WO 2020/117548, WO 2018/210820

SEQUENCE LISTING

SEQ ID NO: 1
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFT
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGDVTMNLTTKVANDTEAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVEDMWN
RSKNGNTEDYGKWFGQAIAGDNAVLGGDKDETWAKEDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKT
SEQ ID NO: 2
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFT
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGDVTMNLTTKVANDTEAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVEDMWN
RSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKTVRSFYYSRNYYVKTGNKPILPSDVEVRYSDGTSDRQNVTWDAVSDDQIAKAGSFSVAGTVAGQKISVRVTMIDEIGAL
SEQ ID NO: 3
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFT
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGDVTMNLTTKVANDTEAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVEDMWN
RSKNGNTEDYGKWFGQAIAGDNAVLGGDKDETWAKEDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSA
STPVGTPAVLPGSRPAVLPDGTVTSANFAVHWTKPADTVYNTAGTVKVPGTATVFGKEFKVTATIRVQ
SEQ ID NO: 4
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFT
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGDVTMNLTTKVANDTEAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVEDMWN
RSKNGNTEDYGKWFGQAIAGDNAVLGGDKDETWAKEDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSA
STPVGTPAVLPGSRPAVLPDGTVTSANFAVHWTKPADTVYNTAGTVKVPGTATVFGKEFKVTATIRVQRSQVTIGSSVSGNALRLTQNIPADKQSDT
LDAIKDGSTTVDANTGGGANPSAWTNWAYSKAGHNTAEITFEYATEQQLGQIVMYFERDSNAVRFPDAGKTKIQI
SEQ ID NO: 5
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFT
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGDVTMNLTTKVANDTEAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWN
RSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSA
STPVGTPAVLPGSRPAVLPDGTVTSANFAVHWTKPADTVYNTAGTVKVPGTATVFGKEFKVTATIRVQRSQVTIGSSVSGNALRLTQNIPADKQSDT
LDAIKDGSTTVDANTGGGANPSAWTNWAYSKAGHNTAEITFEYATEQQLGQIVMYFFRDSNAVRFPDAGKTKIQISADGKNWTDLAATETIAAQESS
DRVKPYTYDFAPVGATFVKVTVTNADTTTPSGVVCAGLTEIELKTAT
SEQ ID NO: 6
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFT
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGDVTMNLTTKVANDTEAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVEDMWN
RSKNGNTEDYGKWFGQAIAGDNAVLGGDKDETWAKEDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSA
STPVGTPAVLPGSRPAVLPDGTVTSANFAVHWTKPADTVYNTAGTVKVPGTATVFGKEFKVTATIRVQRSQVTIGSSVSGNALRLTQNIPADKQSDT
LDAIKDGSTTVDANTGGGANPSAWTNWAYSKAGHNTAEITFEYATEQQLGQIVMYFFRDSNAVRFPDAGKTKIQISADGKNWTDLAATETIAAQESS
DRVKPYTYDFAPVGATFVKVTVTNADTTTPSGVVCAGLTEIELKTATSKFVTNTSAALSSLTVNGTKVSDSVLAAGSYNTPAIIADVKAEGEGNASV
TVLPAHDNVIRVITESEDHVTRKTFTINLGTEQEF
SEQ ID NO: 7
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSET
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGNVTMNLTTKVANDTKAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVEDMWN
RSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGADKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSA
STPVGTPAVLPGSRPAVLPDGTVTSANFAVDWTKPADTVYNTAGTVKVPGTATVFGKEFKVTATIRVQRSQVTIGSSVSGNALRLTQNIPADKQSDT
LDAIKDGSTTVDANTGGGANPSAWTNWAYSKAGHNTAEITFEYATEQQLGQIVMYFERDSNAVRFPDAGKTKIQISADGKNWTDLAATETIAAQESS
ERVKPYTYDFAPVGATFVKVTVTNADTTTPSGVVCAGLTEIELKTATSKFVTNTSAALSSLTVNGTKVSDSVLAAGSYNTPAIIADVKAEGEGNASV
TVLPAHDNVIRVITESEDHVTRKTFTINLGTEQEFPADSDERD
SEQ ID NO: 8
MKKPLGKIVASTALLISVAFSSSIASAAVEDATRSDSTTQMSSTPEVAYSSAVDSKQNRTSDFDANWKFMLSDSVQAQDPAFDDSAWQQVDLPHDYS
ITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRW
YSGSGIYRDVTLTVTDGVHVGNNGVAIKTPSLATQNGGDVTMNLTTKVANDTEAAANITLKQTVFPKGGKTDAAIGTVTTASKSIAAGASADVTSTI
TAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTT
HNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVS
GFPATSAKLVAWTKAADSTRPMTYGDNKIKANWNESNTMGDNLTANGGVVGTNYSDGANYDKIRTTHPSWAIYGSETASAINSRGIYNRTTGGAOSS
DKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHI
LPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENN
RLIPEGSTEGNASVTTTGKAAKLKADADRKTITADGKDLSYIEVDVTDANGHIVPDAANRVTFDVKGAGKLVGVDNGSSPDHDSYQADNRKAFSGKV
LAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSES
VAGTVAGQKISVRVTMIDEIGALLNYSASTPVGTPAVLPGSRPAVLPDGTVTSANFAVHWTKPADTVYNTAGTVKVPGTATVFGKEFKVTATIRVQR
SQVTIGSSVSGNALRLTQNIPADKQSDTLDAIKDGSTTVDANTGGGANPSAWTNWAYSKAGHNTAEITFEYATEQQLGQIVMYFERDSNAVRFPDAG
KTKIQISADGKNWTDLAATETIAAQESSDRVKPYTYDFAPVGATFVKVTVTNADTTTPSGVVCAGLTEIELKTATSKFVTNTSAALSSLTVNGTKVS
DSVLAAGSYNTPAIIADVKAEGEGNASVTVLPAHDNVIRVITESEDHVTRKTFTINLGTEQEFPADSDERDYPAADMTVTVGSEQTSGTATEGPKKF
AVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKA
VHTYADSGNDKFMSASEIRLRKAVDTTDISGATVTVPAKLTVDRVDADHPATFATKDVTVTLGDATLRYGVDYLLDYAGNTAVGKATVTVRGIDKYS
GTVAKTFTIELKNAPAPEPTLTSVSVKTKPSKLTYVVGDAFDPAGLVLQLNYDDDSTGTVTWNTQTAGDFTFKPALDAKLKVTDKTVTVTYQGKSAV
IDITVSQPAPTVSKTDLDKAIKAIEAKNPDSSKYTADSWKTFADAMAHAKAVIADDSATQQDVDNALKALTDAYAGLTEKTPEPAPVSKSELDKKIK
AIEAEKLDGSKYTAESWKAFETALAHAKAVIASDSATQQNVDAALGALTSARDGLTEKGEVKPDPKPEPGTVDKAALDKAVKKVEAEKLDGSKYTAD
SWKAFETALAHAKAVIGNANSTQFDIDNALSMLNDARAALKEKPGRIIAIIDGSALSKTGASVAIIASVAAAMLAVGAGVMALRRKRS
SEQ ID NO: 9
MKKPLGKIVASTALLISVAFSSSIASAIEDATRSDSTTQMSSTPEVAYSSAVDSKQNRTSDFDANWKFMLSDSVQAQDPAFDDSAWQQVDLPHDYSI
TQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWY
SGSGIYRDVTLTVTDGVHVGNNGVAIKTPSLATQNGGDVTMNLTTKVANDTEAAANITLKQTVFPKGGKTDAAIGTVTTASKSIAAGASADVTSTIT
AASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTH
NPAAKALIDVCNEKGVLVVEEVFDMWNRSKNGNTEDYGKWFGQAIAGDNAVLGGDKDETWAKEDLTSTINRDRNAPSVIMWSLGNEMMEGISGSVSG
FSATSAKLVAWTKAADSTRPMTYGDNKIKANWNESNTMGDNLTANGGVVGTNYSDGANYDKIRTTHPSWAIYGSETASAINSRGIYNRTTGGAQSSD
KQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHIL
PAWNENVVAKGSGNNVPVVVYTDAAKVKLYFTPKGSTEQRLIGEKSFTKKTTAAGYTYQVYEGSDKDSTAHKNMYLTWNVPWAEGTISAEAYDENNR
LIPEGSTEGNASVTTTGKAAKLKADADRKTITADGKDLSYIEVDVTDANGHIVPDAANRVTFDVKGAGKLVGVDNGSSPDHDSYQADNRKAFSGKVL
AIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSV
AGTVAGQKISVRVTMIDEIGALLNYSASTPVGTPAVLPGSRPAVLPDGTVTSANFAVHWTKPADTVYNTAGTVKVPGTATVFGKEFKVTATIRVQRS
QVTIGSSVSGNALRLTQNIPADKQSDTLDAIKDGSTTVDANTGGGANPSAWTNWAYSKAGHNTAEITFEYATEQQLGQIVMYFERDSNAVRFPDAGK
TKIQISADGKNWTDLAATETIAAQESSDRVKPYTYDFAPVGATFVRVTVTNADTTTPSGVVCAGLTEIELKTATSKFVANTSAALSSLTVNGTKVSD
SVLAAGSYNTPAIIADVKAEGEGNASVTVLPAHDNVIRVITESEDHVTRKTFTINLGTEQEFPADSDERDQHQHQHQHQQ
SEQ ID NO: 10
MAVRRLGGRIVAFAATVALSIPLGLLTNSAWAVEDATRSDSTTQMSSTPEVVYSSAVDSKQNRTSDFDANWKFMLSDSVQAQDPAFDDSAWQQVDLP
HDYSITQKYSQSNEAESAYLPGGTGWYRKSFTIDRDLAGKRIAINFDGVYMNATVWFNGVKLGTHPYGYSPFSFDLTGNAKFGGENTIVVKVENRLP
SSRWYSGSGIYRDVTLTVTDGVHVGNNGVAIKTPSLATQNGGDVTMNLTTKVANDTEAAANITLKQTVFPKGGKTDAAIGTVTTASKSIAAGASADV
TSTITAASPKLWSIKNPNLYTVRTEVLNGGKVLDTYDTEYGFRWTGFDATSGFSLNGEKVKLKGVSMHHDQGSLGAVANRRAIERQVEILQKMGVNS
IRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGIS
GSVSGFPATSAKLVAWTKAADSTRPMTYGDNKIKANWNESNTMGDNLTANGGVVGTNYSDGANYDKIRTTHPSWAIYGSETASAINSRGIYNRTTGG
AQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVH
TLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAY
DENNRLIPEGSTEGNASVTTTGKAAKLKADADRKTITADGKDLSYIEVDVTDANGHIVPDAANRVTFDVKGAGKLVGVDNGSSPDHDSYQADNRKAF
SGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKA
GSFSVAGTVAGQKISVRVTMIDEIGALLNYSASTPVGTPAVLPGSRPAVLPDGTVTSANFAVHWTKPADTVYNTAGTVKVPGTATVFGKEFKVTATI
RVQRSQVTIGSSVSGNALRLTQNIPADKQSDTLDAIKDGSTTVDANTGGGANPSAWTNWAYSKAGHNTAEITFEYATEQQLGQIVMYFERDSNAVRE
PDAGKTKIQISADGKNWTDLAATETIAAQESSDRVKPYTYDFAPVGATFVKVTVTNADTTTPSGVVCAGLTEIELKTATSKFVTNTSAALSSLTVNG
TKVSDSVLAAGSYNTPAIIADVKAEGEGNASVTVLPAHDNVIRVITESEDHVTRKTFTINLGTEQEFPADSDERDYPAADMTVTVGSEQTSGTATEG
PKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHV
RLKAVHTYADSGNDKFMSASEIRLRKAVDTTDISGATVTVPAKLTVDRVDADHPATFATKDVTVTLGDATLRYGVDYLLDYAGNTAVGKATVTVRGI
DKYSGTVAKTFTIELKNAPAPEPTLTSVSVKTKPSKLTYVVGDAFDPAGLVLQHDRQADRPPQPLVGEQADERGLTCGTRCDRVEQLRKHENREAHR
TGLDHLEFVGAADGAVGEQATFKVHVHADQGDGRHDDADERDIDPHVPVDHAVGELARAACHHVIGLRVDTHRLKASGFQIPADDMAEIDRITGFHR
FERHVG
SEQ ID NO: 11
MAVRRLGGRIVAFAATVALSIPLGLLTNSAWAVEDATRSDSTTQMSSTPEVVYSSAVDSKQNRTSDFDANWKFMLSDSVQAQDPAFDDSAWQQVDLP
HDYSITQKYSQSNEAESAYLPGGTGWYRKSFTIDRDLAGKRIAINFDGVYMNATVWFNGVKLGTHPYGYSPFSFDLTGNAKFGGENTIVVKVENRLP
SSRWYSGSGIYRDVTLTVTDGVHVGNNGVAIKTPSLATQNGGNVTMNLTTKVANDTKAAANITLKQTVFPKGGKTDAAIGTVTTASKSIAAGASADV
TSTITAASPKLWSIKNPNLYTVRTEVLNGGKVLDTYDTEYGFRWTGFDATSGFSLNGEKVKLKGVSMHHDQGSLGAVANRRAIERQVEILQKMGVNS
IRTTHNPAAKALIDVCNEKGVLVVEEVFDMWNRSKNGNTEDYGKWFGQAIAGDNAVLGGDKDETWAKEDLTSTINRDRNAPSVIMWSLGNEMMEGIS
GSVSGFPATSAKLVAWTKAADSTRPMTYGDNKIKANWNESNTMGDNLTANGGVVGTNYSDGANYDKIRTTHPSWAIYGSETASAINSRGIYNRTTGG
AQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVH
TLHILPAWNENVVAKGSGNNVPVVVYTDAAKVKLYFTPKGSTEKRLIGEKSFTKKTTAAGYTYQVYEGADKDSTAHKNMYLTWNVPWAEGTISAEAY
DENNRLIPEGSTEGNASVTTTGKAAKLKADADRKTITADGKDLSYIEVDVTDANGHIVPDAANRVTFDVKGAGKLVGVDNGSSPDHDSYQADNRKAF
SGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKA
GSFSVAGTVAGQKISVRVTMIDEIGALLNYSASTPVGTPAVLPGSRPAVLPDGTVTSANFAVDWTKPADTVYNTAGTVKVPGTATVFGKEFKVTATI
RVQRSQVTIGSSVSGNALRLTQNIPADKQSDTLDAIKDGSTTVDANTGGGANPSAWTNWAYSKAGHNTAEITFEYATEQQLGQIVMYFERDSNAVRE
PDAGKTKIQISADGKNWTDLAATETIAAQESSDRVKPYTYDFAPVGATFVKVTVTNADTTTPSGVVCAGLTEIELKTATSKFVTNTSAALSSLTVNG
TKVSDSVLAAGSYNTPAIIADVKAEGEGNASVTVLPAHDNVIRVITESEDHVTRKTFTINLGTEQEFPADSDERDYPAADMTVTAGSEQTSGTATEG
PKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHV
RLKAVHTYADSGNDKFMSASEIRLRKAVDTTDISGATVTVPAKLTVDRVDADHPATFATKDVTVTLGDATLRYGVDYLLDYAGNTAVGKATVTVRGI
DKYSGTVAKTFTIELKNAPAPEPTLTSVSVKTKPSKLTYVVGDAFDPAGLVLQLNYDDDSTGTVTWNTQTAGDFTFKPALDAKLKVTDKTVTVTYQG
KSAVIDITVSQPAPTVSKTDLDKAIKAIEAKNPDSSKYTADSWKTFADAMAHAKAVIADDSATQQDVDKALKALTDAYAGLTEKTPEPAPVSKSELD
KKIKAIEAEKLDGSKYTAESWKAFETALAHAKAVIASDSATQQDVDAALGALTSARDGLTEKGEVKPDPKPEPGTVDKAALDKAVKKVEAEKLDGSK
YTADSWKAFETALAHAKAVIGNANSTQFDIDNALSMLNDARAALKEKPGRIIAIIDGGALSKTGASVAIIASVAAAMKAVGAGVMALRPPKW
SEQ ID NO: 12
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFT
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGDVTMNLTTKVANDTEAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVEDMWN
RSKNGNTEDYGKWFGQAIAGDNAVLGGDKDETWAKEDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKT
SEQ ID NO: 13
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFT
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGDVTMNLTTKVANDTEAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVEDMWN
RSKNGNTEDYGKWFGQAIAGDNAVLGGDKDETWAKEDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKTVRSFYYSRNYYVKTGNKPILPSDVEVRYSDGTSDRQNVTWDAVSDDQIAKAGSFSVAGTVAGQKISVRVTMIDEIGAL
SEQ ID NO: 14
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFT
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGDVTMNLTTKVANDTEAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVEDMWN
RSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSA
STPVGTPAVLPGSRPAVLPDGTVTSANFAVHWTKPADTVYNTAGTVKVPGTATVFGKEFKVTATIRVQ
SEQ ID NO: 15
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFT
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGDVTMNLTTKVANDTEAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWN
RSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSA
STPVGTPAVLPGSRPAVLPDGTVTSANFAVHWTKPADTVYNTAGTVKVPGTATVFGKEFKVTATIRVQRSQVTIGSSVSGNALRLTQNIPADKQSDT
LDAIKDGSTTVDANTGGGANPSAWTNWAYSKAGHNTAEITFEYATEQQLGQIVMYFERDSNAVRFPDAGKTKIQI
SEQ ID NO: 16
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFT
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGDVTMNLTTKVANDTEAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVEDMWN
RSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSA
STPVGTPAVLPGSRPAVLPDGTVTSANFAVHWTKPADTVYNTAGTVKVPGTATVFGKEFKVTATIRVQRSQVTIGSSVSGNALRLTQNIPADKQSDT
LDAIKDGSTTVDANTGGGANPSAWTNWAYSKAGHNTAEITFEYATEQQLGQIVMYFERDSNAVRFPDAGKTKIQISADGKNWTDLAATETIAAQESS
DRVKPYTYDFAPVGATFVKVTVTNADTTTPSGVVCAGLTEIELKTAT
SEQ ID NO: 17
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFT
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGDVTMNLTTKVANDTEAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVEDMWN
RSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSA
STPVGTPAVLPGSRPAVLPDGTVTSANFAVHWTKPADTVYNTAGTVKVPGTATVFGKEFKVTATIRVQRSQVTIGSSVSGNALRLTQNIPADKQSDT
LDAIKDGSTTVDANTGGGANPSAWTNWAYSKAGHNTAEITFEYATEQQLGQIVMYFERDSNAVRFPDAGKTKIQISADGKNWTDLAATETIAAQESS
DRVKPYTYDFAPVGATFVKVTVTNADTTTPSGVVCAGLTEIELKTATSKFVTNTSAALSSLTVNGTKVSDSVLAAGSYNTPAIIADVKAEGEGNASV
TVLPAHDNVIRVITESEDHVTRKTFTINLGTEQEF
SEQ ID NO: 18
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFT
IDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIK
TPSLATQNGGDVTMNLTTKVANDTEAAANITLKQTVFPKGGKTDAAIGTVTTASKSIAAGASADVTSTITAASPKLWSIKNPNLYTVRTEVLNGGKV
LDTYDTEYGFRWTGFDATSGFSLNGEKVKLKGVSMHHDQGSLGAVANRRAIERQVEILQKMGVNSIRTTHNPAAKALIDVCNEKGVLVVEEVEDMWN
RSKNGNTEDYGKWFGQAIAGDNAVLGGDKDETWAKEDLTSTINRDRNAPSVIMWSLGNEMMEGISGSVSGFPATSAKLVAWTKAADSTRPMTYGDNK
IKANWNESNTMGDNLTANGGVVGTNYSDGANYDKIRTTHPSWAIYGSETASAINSRGIYNRTTGGAQSSDKQLTSYDNSAVGWGAVASSAWYDVVQR
DFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKV
KLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADAD
RKTITADGKDLSYIEVDVTDANGHIVPDAANRVTFDVKGAGKLVGVDNGSSPDHDSYQADNRKAFSGKVLAIVQSTKEAGEITVTAKADGLQSSTVK
IATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSA
STPVGTPAVLPGSRPAVLPDGTVTSANFAVHWTKPADTVYNTAGTVKVPGTATVFGKEFKVTATIRVQRSQVTIGSSVSGNALRLTQNIPADKQSDT
LDAIKDGSTTVDANTGGGANPSAWTNWAYSKAGHNTAEITFEYATEQQLGQIVMYFFRDSNAVRFPDAGKTKIQISADGKNWTDLAATETIAAQESS
DRVKPYTYDFAPVGATFVKVTVTNADTTTPSGVVCAGLTEIELKTATSKFVTNTSAALSSLTVNGTKVSDSVLAAGSYNTPAIIADVKAEGEGNASV
TVLPAHDNVIRVITESEDHVTRKTFTINLGTEQEFPADSDERDYPAADMTVTVGSEQTSGTATEGPKKFAVDGNTSTYWHSNWTPTTVNDLWIAFEL
QKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTD
ISGATVTVPAKLTVDRVDADHPATFATKDVTVTLGDATLRYGVDYLLDYAGNTAVGKATVTVRGIDKYSGTVAKTFTIELKNAPAPEPTLTSVSVKT
KPSKLTYVVGDAFDPAGLVLQHDRQADRPPQPLVGEQADERGLTCGTRCDRVEQLRKHENREAHRTGLDHLEFVGAADGAVGEQATFKVHVHADQGD
GRHDDADERDIDPHVPVDHAVGELARAACHHVIGLRVDTHRLKASGFQIPADDMAEIDRITGFHRFERHVG

### LIST OF EMBODIMENTS IN CLAIM FORMAT

1. Method for producing a fermented milk-based product comprising galacto-oligosaccharides, wherein the method comprises the steps of:
   (a) providing a milk-based substrate comprising lactose;
   (b) treating the milk-based substrate with a beta-galactosidase enzyme to generate galacto-oligosaccharides followed by fermenting the milk-based substrate treated with the beta-galactosidase enzyme until a pH below 5 is reached, preferably at 43° C.; or alternatively fermenting the milk-based substrate until a pH below 5 is reached, preferably at 43° C., followed by treating the fermented milk-based substrate with a beta-galactosidase enzyme to generate galacto-oligosaccharides;
   (c) inactivating the beta-galactosidase enzyme by submitting the fermented milk-based substrate comprising galacto-oligosaccharides having a pH below 5 to a temperature below 95° C., preferably from 70° C. to less than 95° C. or from 70° C. to 90° C., preferably from 70° C. to 85° C. or from 72° C. to 80° C., more preferably from 70° C. to 75° C., whereby producing the fermented milk-based product comprising galacto-oligosaccharides.
2. Method according to the previous embodiment, wherein the galacto-oligosacharides comprised in the fermented milk-based product, such as yogurt or yogurt product, are stable for at least 5 days at a temperature of 15-37° C., preferably 15-30° C., more preferably 18-24° C., and at a pressure of 1 atm.
3. Method according to any of the previous embodiments, wherein the galacto-oligosacharides comprised in the fermented milk-based product, such as yogurt or yogurt product, are stable for at least 10 days at a temperature of 15-37° C., preferably 15-30° C., more preferably 18-24° C., and at a pressure of 1 atm.
4. Method according to any of the previous embodiments, wherein the galacto-oligosacharides comprised in the fermented milk-based product, such as yogurt or yogurt product, are stable for at least 15 days at a temperature of 15-37° C., preferably 15-30° C., more preferably 18-24° C., and at a pressure of 1 atm.
5. Method according to any of the previous embodiments, wherein the galacto-oligosacharides comprised in the fermented milk-based product, such as yogurt or yogurt product, are stable for at least 21 days at a temperature of 15-37° C., preferably 15-30° C., more preferably 18-24° C., and at a pressure of 1 atm.
6. Method according to any of the previous embodiments, wherein the galacto-oligosacharides comprised in the fermented milk-based product, such as yogurt or yogurt product, are stable for at least 90 days at a temperature of 15-37° C., preferably 15-30° C., more preferably 18-24° C., and at a pressure of 1 atm.
7. Method according to any of the previous embodiments, wherein the galacto-oligosacharides comprised in the fermented milk-based product, such as yogurt or yogurt product, are stable for at least 180 days at a temperature of 15-37° C., preferably 15-30° C., more preferably 18-24° C., and at a pressure of 1 atm.
8. Method according to any of the previous embodiments, wherein the galacto-oligosacharides comprised in the fermented milk-based product, such as yogurt or yogurt product, are stable for 1 day to 250 days, more preferably for 1 day to 200 days, even more preferably for 1 day to 180 days, at a temperature of 15-37° C., preferably 15-30° C., more preferably 18-24° C., and at a pressure of 1 atm.
9. Method according to any of the previous embodiments, wherein the method comprises a further step of storing the fermented milk-based product comprising galacto-oligosaccharides at a temperature of 15-37° C., preferably 15-30° C., more preferably 18-24° C., and at a pressure of 1 atm.
10. Method according to any of the previous embodiments, wherein the method is carried out by using a milk-based substrate comprising at least 1% W_{lactose}/W_{milk-based substrate}, preferably 1-60% W_{lactose}/W_{milk-based substrate}, more preferably 2-50% W_{lactose}/W_{milk-based substrate}, even more preferably 3-40% W_{lactose}/W_{milk-based substrate}.
11. Method according to any of the previous embodiments, wherein the method is carried out by using as milk-based substrate milk selected from: cow milk, sheep milk, goat milk, buffalo milk, camel milk, pasteurized milk, raw milk, filtered milk, or combinations thereof.
12. Method according to any of the previous embodiments, wherein the step of treating the milk-based substrate with a beta-galactosidase enzyme to generate galacto-oligosaccharides is carried out for 1-24 hours, preferably 3-22 hours, more preferably 5-18 hours and/or at a temperature of 15-60° C., preferably 25-45° C., more preferably at 43° C.
13. Method according to any of the previous embodiments, wherein the step of treating the milk-based substrate with a beta-galactosidase enzyme is carried out with at least 0.1 g/L_{milk-based substrate} of beta-galactosidase enzyme or at most 10 g/L_{milk-based substrate} of beta-galactosidase enzyme or with less than 10 g/L_{milk-based substrate} of beta-galactosidase enzyme, preferably with 0.1-10 g/L_{milk-based substrate} of beta-galactosidase enzyme or 0.1-9 g/L_{milk-based substrate} of beta-galactosidase enzyme or 0.1-8 g/L_{milk-based substrate} of beta-galactosidase enzyme, more preferably with 0.2-7 g/L_{milk-based substrate} Of beta-galactosidase enzyme or 0.3-8 g/L_{milk-based substrate} of beta-galactosidase enzyme, even more preferably with 0.3-5 g/L_{milk-based substrate} of beta-galactosidase enzyme or 0.3-3 g/L_{milk-based substrate} of beta-galactosidase enzyme or 0.5-1 g/L_{milk-based substrate} of beta-galactosidase enzyme. Additionally, “g/L_{milk-based substrate} of beta-galactosidase enzyme” and “g_{beta-galactosidase enzyme}/L_{milk-based substrate}” have the meaning and are, in the entire disclosure interchangeable.
14. Method according to any of the previous embodiments, wherein the method is carried out using a Bifidobacterium beta-galactosidase enzyme, preferably wherein the beta-galactosidase enzyme is Bifidobacterium bifidum beta-galactosidase, more preferably wherein the beta-galactosidase enzyme has at least 80% or at least 85% or at least 90% or at least 95% or at least 96% or at least 97% or at least 98% or at least 99% or 100% sequence identity to SEQ ID NOs 1, 2, 3, 4, 5, 6, 7, 8, 9, 10, 11, 12, 13, 14, or 15 or a sequence, even more preferably wherein the sequence is SEQ ID NO 1 or SEQ ID NO 7.
15. Method according to any of the previous embodiments, wherein the method is carried out by using a starter culture for achieving the fermentation of the milk-based substrate or milk-based substrate comprising galacto-oligosaccharides.
16. Method according to any of the previous embodiments, wherein the step of inactivating the beta-galactosidase enzyme is be carried out at a pH below 5, such as for example at a pH between 3.4 to 4.5, preferably between 3.6 to 4.4, more preferably 3.8 to 4.3, and from 70° C. to less than 95° C., preferably from 70° C. to 90° C. or from 70° C. to 85° C. or preferably from 72° C. to 80° C., more preferably from 70° C. to 75° C.
17. Method according to any of the previous embodiments, wherein the step of inactivating the beta-galactosidase enzyme is carried out for about 5 seconds to 30 minutes, or for about 10 seconds to 20 minutes, or for about 15 seconds to 15 minutes, or for about 20 seconds to about 5 minutes, or for about 5 seconds to about 20 seconds or for about 5 seconds to 10 seconds or for about 10 to 20 seconds.
18. Method according to any of the previous embodiments, wherein the step of inactivating the beta-galactosidase enzyme is carried from 70° C. to 75° C. for 5 seconds to 60 seconds, preferably from 70° C. to 75° C. for 10 seconds to 40 seconds, more preferably from 70° C. to 75° C. for 15 seconds to 20 seconds, more preferably 72° C. for 20 seconds or 75° C. for 20 seconds.
19. Method according to any of the previous embodiments, wherein the step of inactivating the beta-galactosidase may be carried out from at 70° C. to 75° C. for 10 seconds to 60 seconds and at a pH below 5, preferably from 70° C. to 75° C. for 20 seconds to 60 seconds and at a pH below 5, more preferably from 72° C. for 20 seconds and at a pH of 4.3 or from 75° C. for 20 seconds and at a pH of 4.3.
20. Method according to any of the previous embodiments, wherein the method is a method for producing a fermented milk-based product, wherein said product is a yoghurt or an ambient yogurt or a yogurt product or a ambient yogurt product or a post-pasteurized yogurt, buttermilk, creme fraiche, quark, fromage frais, yakult or skyr; or wherein the fermented milk product is a stirred fermented milk product or drinking fermented milk product, preferably wherein the stirred fermented milk product is a stirred yogurt or the drinking fermented milk product is drinking yogurt, more preferably wherein the stirred yogurt is a post-pasteurization stirred yogurt or the drinking yogurt is an post-pasteurization drinking yogurt.
21. Method according to any of the previous embodiments, wherein the fermented milk-based product, such as yogurt or yogurt product or ambient yogurt or ambient yogurt product, comprises at least 0.5% W_{galacto-oligosaccharides}/W_{fermented milk-based product}, preferably 0.5-4% W_{galacto-oligosaccharides}/W_{fermented milk-based product}, more preferably 1-3% W_{galacto-oligosaccharides}/W_{fermented milk-based product}, even more preferably 1.5-2.5% W_{galacto-oligosaccharides}/W_{fermented milk-based product}.
22. Method according to any of the previous embodiments, wherein the fermented milk-based product, such as yogurt or yogurt product or ambient yogurt or ambient yogurt product, comprises at least 0.1% W_{galacto-oligosaccharides with DP>2}/W_{fermented milk-based product}, preferably 0.2-3% W_{galacto-oligosaccharides with DP>2}/W_{fermented milk-based product}, more preferably 0.3-2% W_{galacto-oligosaccharides with DP>2}/W_{fermented milk-based product}, even more preferably 0.4-1% W_{galacto-oligosaccharides with DP>2}/W_{fermented milk-based product}.
23. Fermented milk-based product comprising galacto-oligosaccharides obtained by the method of any of the embodiments 1-22.
24. Yogurt or yogurt product comprising galacto-oligosaccharides obtained by the method of any of the embodiments 1-22, preferably wherein the yogurt or yogurt product is a stirred yogurt or the drinking fermented milk product is drinking yogurt, more preferably wherein the stirred yogurt is a post-pasteurization stirred yogurt or the drinking yogurt is an post-pasteurization drinking yogurt.
25. Fermented milk-based product comprising galacto-oligosaccharides, in particular a yogurt or an ambient yogurt or a yogurt product or a ambient yogurt product or a post-pasteurized yogurt, wherein the pH of the fermented milk-based product is below 5, preferably between 3.4 to 4.5, more preferably between 3.6 to 4.4, even more preferably 3.8 to 4.3; and wherein the fermented milk-based product is stored at a temperature of 15-37° C., preferably 15-30° C., more preferably 18-24° C. and at a pressure of 1 atm
26. Fermented milk-based product according to embodiment 25, further comprising an inactivated beta-galactosidase enzyme, preferably wherein the inactivated beta-galactosidase enzyme is a Bifidobacterium beta-galactosidase enzyme or wherein the inactivated beta-galactosidase enzyme is a Bifidobacterium bifidum beta-galactosidase, more preferably wherein the inactivated beta-galactosidase enzyme has at least 80% or at least 85% or at least 90% or at least 95% or at least 96% or at least 97% or at least 98% or at least 99% or 100% sequence identity to SEQ ID NOs 1, 2, 3, 4, 5, 6, 7, 8, 9, 10, 11, 12, 13, 14, or 15 or a sequence, even more preferably wherein the sequence is SEQ ID NO 1 or SEQ ID NO 7.
27. Fermented milk-based product according to any of the embodiments 25-26, comprising at least 0.5% W_{galacto-oligosaccharides}/W_{fermented milk-based product}, more preferably 0.5-4% W_{galacto-oligosaccharides}/W_{fermented milk-based product}, even more preferably 1-3% W_{galacto-oligosaccharides}/W_{fermented milk-based product} or 1.5-2.5% W_{galacto-oligosaccharides}/W_{fermented milk-based product}.
28. Fermented milk-based product according to any of the embodiments 25-27, wherein the galacto-oligosaccharides comprised in the fermented milk-based product have a DP of more than 2 or at least 3; preferably the galacto-oligosaccharides comprise or include lactosucrose.
29. Fermented milk-based product according to any of the embodiments 25-28, comprising at least 0.1% W_{galacto-oligosaccharides with DP>2}/W_{fermented milk-based product}, preferably 0.2-3% W_{galacto-oligosaccharides with DP>2}/W_{fermented milk-based product}, more preferably 0.3-2% W_{galacto-oligosaccharides with DP>2}/W_{fermented milk-based product}, even more preferably 0.4-1% W_{galacto-oligosaccharides with DP>2}/W_{fermented milk-based product}.
30. Fermented milk-based product according to any of the embodiments 25-29, wherein the fermented milk-based product comprising galacto-oligosaccharides is a yoghurt, a yogurt product, buttermilk, creme fraiche, quark, fromage frais, yakult or skyr.
31. Fermented milk-based product according to any of the embodiments 25-30, wherein the fermented milk product is a stirred fermented milk product, preferably the stirred fermented milk product is a stirred yogurt or a stirred yogurt product, more preferably the stirred yogurt is a post-pasteurization stirred yogurt or a post-pasteurization stirred yogurt product; or the fermented milk product is a drinking fermented milk product, preferably the drinking fermented milk product is a drinking yogurt or a drinking yogurt product, more preferably drinking yogurt is a post-pasteurization drinking yogurt or a post-pasteurization drinking yogurt product.
32. Fermented milk-based product according to any of the embodiments 25-31, wherein the galacto-oligosacharides comprised in the fermented milk-based product are stable for at least 5 days at a temperature of 15-37° C., preferably 15-30° C., more preferably 18-24° C., and at a pressure of 1 atm, preferably for at least 10 days, more preferably for at least 15 days, even more preferably for at least 21 days.
33. Method for inactivating a beta-galactosidase enzyme comprising the step of submitting the beta-galactosidase enzyme to a temperature below 95° C. at a pH below 5, preferably comprising the step of submitting the beta-galactosidase enzyme to a temperature from 70° C. to 85° C. and a pH below 5.
34. Method according to the previous embodiment 33, wherein the method for inactivating a beta-galactosidase enzyme is carried out at a temperature from 70° C. to less than 95° C., preferably from 70° C. to 90° C., preferably from 70° C. to 85° C. or more preferably from 72° C. to 80° C., even more preferably from 70° C. to 75° C.
35. Method according to any of the previous embodiments 33-34, wherein the method is carried out at a pH between 3.4 to 4.5, preferably between 3.6 to 4.4, more preferably 3.8 to 4.3.
36. Method according to any of the previous embodiments 33-35, wherein the method is carried out at a temperature from 70° C. to less than 95° C., preferably from 70° C. to 90° C., preferably from 70° C. to 85° C. or more preferably from 72° C. to 80° C., even more preferably from 70° C. to 75° C., and at a pH between 3.4 to 4.5, preferably between 3.6 to 4.4, more preferably 3.8 to 4.3.
37. Method according to any of the previous embodiments 33-36, wherein the method is carried out for about 5 seconds to 30 minutes, or for about 10 seconds to 20 minutes, or for about 15 seconds to 15 minutes, or for about 20 seconds to about 5 minutes, preferably the method is be carried out for about 5 seconds to about 20 seconds or for about 5 seconds to 10 seconds or for about 10 to 20 seconds.
38. Method according to any of the previous embodiments 33-37, wherein the beta-galactosidase enzyme is a Bifidobacterium bifidum beta-galactosidase, more preferably wherein the beta-galactosidase enzyme has at least 80%, at least 85%, at least 90%, at least 95%, at least 96%, at least 97%, at least 98%, at least 99% or 100% sequence identity to SEQ ID NOs 1, 2, 3, 4, 5, 6, 7, 8, 9, 10, 11, 12, 13, 14, or 15 or a sequence, even more preferably wherein the sequence is SEQ ID NO 1 or SEQ ID NO 7.

1. A method for producing a yogurt comprising galacto-oligosaccharides, wherein the method comprises
(a) treating a milk-based substrate comprising at least 1% w/w lactose with a beta-galactosidase enzyme to generate galacto-oligosaccharides;
(b) before or after said treating, fermenting the milk-based substrate until a pH below 5 is reached; and
(c) inactivating the beta-galactosidase enzyme by subjecting the fermented milk-based substrate comprising galacto-oligosaccharides having a pH below 5 to heat treatment at a temperature from 70° C. to 85° C.,
thereby producing the yogurt comprising galacto-oligosaccharides.

2. The method according to claim 1, further comprising storing the yogurt at a temperature of 15-37° C. and at a pressure of 1 atm.

3. The method according to claim 1, wherein the galacto-oligosacharides comprised in the yogurt are stable for at least 5 days when the yogurt is stored at a temperature of 15-37° C. and at a pressure of 1 atm.

4. The method according to claim 1, wherein the step of treating the milk-based substrate with a beta-galactosidase enzyme is carried out with from 0.1-10 g enzyme per L milk-based substrate.

5. The method according to claim 1, wherein the beta-galactosidase enzyme is a Bifidobacterium beta-galactosidase.

6. The method according to claim 1, wherein the step of inactivating the beta-galactosidase enzyme is carried out at a temperature from 72° C. to 80° C.

7. The method according to claim 1, wherein the step of inactivating the beta-galactosidase enzyme is carried out at a temperature from 70° C. to 75° C. for 5 seconds to 60 seconds.

8. The method according to claim 1, wherein the step of inactivating the beta-galactosidase is carried out at a temperature from 70° C. to 75° C. for 10 seconds to 60 seconds and at a pH of 4.3.

9. The method according to claim 1, wherein the yogurt is a post-pasteurization stirred yogurt or a post-pasteurization drinking yogurt.

10. The method according to claim 1, wherein the yogurt comprises at least 0.5% w/w galacto-oligosaccharides.

11. A yogurt prepared by a process according to claim 1, comprising at least 0.5% w/w galacto-oligosaccharides, and having a pH below 5.

12. A yogurt comprising at least 0.5% w/w galacto-oligosaccharides and having a pH below 5, further comprising an inactivated beta-galactosidase enzyme.

13. The yogurt according to claim 12, wherein the galacto-oligosaccharides have a polymerization degree of at least 3.

14. The yogurt according to claim 12, wherein the yogurt is a post-pasteurization stirred yogurt or a post-pasteurization drinking yogurt.

15. The yogurt according to claim 12, wherein the galacto-oligosacharides are stable for at least 5 days when the yogurt is stored at a temperature of 15-37° C. and at a pressure of 1 atm.

16. The method according to claim 1, wherein the fermenting is carried out at 43° C.

17. The method according to claim 1, wherein the step of inactivating the beta-galactosidase enzyme is carried out at a temperature from 70° C. to 75° C.

18. The method according to claim 5, wherein the beta-galactosidase enzyme has at least 90% sequence identity to any one of SEQ ID NOs 1-15.

19. The method according to claim 1, wherein the yogurt comprises 0.5-4% w/w galacto-oligosaccharides.
